(19) United States
(12) Patent Application Publication
(10) Pub. No.: US 2014/0363853 A1
(43) Pub. Date: Dec. 11, 2014

(54) NUCLEOTIDE CLONING METHODS

(71) Applicant: Momenta Pharmaceuticals. Inc,, Cambridge, MA (US)

(72) Inventor: James Meador, III, Framingham, MA (US)

(21) Appl. No.: 14/371,947

(22) PCT Filed: Jan. 11, 2013
(86) PCT No.: PCT/US2013/021099

§ 371 Date: Jul. 11, 2014

Related U.S. Application Data
(60) Provisional application No. 61/586,317, filed on Jan. 13, 2012.

(51) Int. Cl. C12N 15/64

(52) U.S. Cl. CPC C12N 15/64
USPC 435/91.41; 435/476

(57) ABSTRACT

Methods of cloning insert sequences into cloning vectors with high efficiency and in the correct orientation are described. In one aspect, the invention features a method of producing a plasmid comprising an insert fragment and a vector fragment in a predetermined orientation. In some embodiments, the method includes cleaving a first nucleotide sequence at a plurality of sites with a first restriction enzyme to generate a first population of nucleotide fragments, the first population of nucleotide fragments comprising insert fragments and non-insert fragments, the insert fragments comprising a non-palindromic overhang at a 5′ end, at a 3′ end, or at both.

### CROSS REFERENCE TO RELATED APPLICATIONS

The present application claims the benefit of U.S. provisional application Ser. No. 61/586,317, filed on Jan. 13, 2012, the contents of which are herein incorporated by reference in their entirety.

### FIELD OF THE INVENTION

The invention relates generally to molecular biology.

### BACKGROUND

Methods of genetic engineering frequently utilize double-stranded DNA constructs made by cloning a target sequence into a vector background, which often requires screening the constructs to ensure the insertion of target sequences in the proper orientation and in the proper translational reading frame. Screening for a correct construct can involve a great deal of time and expense. It would be useful to develop high-efficiency methods of cloning target sequences into vectors and to reduce the need to screen plasmids for the correct orientation of a target sequence cloned into a vector.

### SUMMARY

The invention is based, in part, on the discovery of novel cloning methods that are highly efficient and that enable a high percentage of the cloned sequences to be in the correct orientation. The cloning methods utilize restriction enzymes that produce non-palindromic overhangs within double-stranded sequences of target sequences and vector sequences. Accordingly, the invention features high efficiency cloning methods as well as methods of producing high efficiency cloning vectors.

In one aspect, the invention features a method of producing a plasmid comprising an insert fragment and a vector fragment in a predetermined orientation. In some embodiments, the method includes cleaving a first nucleotide sequence at a plurality of sites with a first restriction enzyme to generate a first population of nucleotide fragments, the first population of nucleotide fragments comprising insert fragments and non-insert fragments, the insert fragments comprising a non-palindromic overhang at a 5′ end, at a 3′ end, or at both. In some embodiments, the method further includes cleaving a second nucleotide sequence with a second restriction enzyme to generate a second population of nucleotide fragments, the second population comprising vector fragments and non-vector fragments, the vector fragments comprising a non-palindromic overhang at a 5′ end, at a 3′ end, or at both. In certain embodiments, the method further includes purifying at least a portion of the vector fragments from the second population. In particular embodiments, the method further includes ligating a plurality of the first population of nucleotide fragments to the purified vector fragments with a ligase, thereby producing a plurality of plasmids; and purifying the plasmids. In some embodiments, at least 95% of a population of microorganisms transformed with the purified plasmids include a plasmid comprising an insert fragment and a vector fragment in a predetermined orientation.

In certain embodiments, at least 90%, at least 91%, at least 92%, at least 93%, at least 94%, at least 95%, at least 96%, at least 97%, at least 98%, at least 99%, or 100% of a population of microorganisms transformed with the purified plasmids include a plasmid comprising an insert fragment and a vector fragment in a predetermined orientation.

In some embodiments, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, about 99% to about 100%, or 100% of a population of microorganisms transformed with the purified plasmids include a plasmid comprising an insert fragment and a vector fragment in a predetermined orientation.

In particular embodiments, the method further includes purifying a plurality of the insert fragments before the ligation step.

In some embodiments, the first restriction enzyme and the second restriction enzyme are the same. In some embodiments, the first restriction enzyme and the second restriction enzyme are different.

In some embodiments, the first nucleotide sequence comprises a PCR product. In some embodiments, the first nucleotide sequence is isolated from a second plasmid.

In certain embodiments, the first nucleotide sequence comprises at least 2, at least 3, at least 4, at least 5, at least 6, at least 7, at least 8, at least 9, or more, internal sites cleaved by the first restriction enzyme. In certain embodiments, the second nucleotide sequence comprises at least 2, at least 3, at least 4, at least 5, at least 6, at least 7, at least 8, at least 9, or more, internal sites cleaved by the second restriction enzyme.

In some embodiments, the first population of nucleotide fragments comprises at least 3, at least 4, at least 5, at least 6, or more, types of insert fragments, wherein each of the 3, 4, 5, 6, or more, types of insert fragment comprises a unique non-palindromic overhang. In some embodiments, the first population of nucleotide fragments comprises at least 3, at least 4, at least 5, at least 6, or more, types of insert fragments, wherein at least 3, at least 4, at least 5, at least 6, or more, types of insert fragment comprises a unique non-palindromic overhang.

In some embodiments, the first nucleotide sequence is not methylated.

In some embodiments, the restriction enzyme is a Type IIS restriction enzyme. In certain embodiments, the restriction enzyme is Bsr I, Bsm I, BsmA I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, Psr I, Ple I, Fau I, Sap I, BspQ I, BspM I, SfaN I, Hga I, Bvb I, Fok I, BceA I, BsmF I, Ksp632 I, Eco31 I, Esp3 I, Aar I, Mly I, or Btr I. In particular embodiments, the restriction enzyme is SapI.

In some embodiments, the non-palindromic overhangs of the insert fragments and the vector fragments independently include 3, 4, 5, 6, 7, 8, 9, or more nucleotides.

In another aspect, the invention features a method of transforming microorganisms with a plasmid comprising an insert fragment and a vector fragment in a predetermined orientation. In some embodiments, the method includes cleaving a first nucleotide sequence at a plurality of sites with a first restriction enzyme to generate a first population of nucleotide fragments, the first population of nucleotide fragments comprising insert fragments and non-insert fragments, the insert fragments comprising a non-palindromic overhang at a 5′ end, at a 3′ end, or at both. In particular embodiments, the method further includes cleaving a second nucleotide sequence with a second restriction enzyme to generate a second population of nucleotide fragments, the second population comprising vector fragments and non-vector fragments, the vector fragments comprising a non-palindromic overhang at a 5′ end, at a 3′ end, or at both. In some embodiments, the method further includes purifying at least a portion of the vector fragments from the second population. In certain embodiments, the method further includes ligating a plurality of the first population of nucleotide fragments to the purified vector fragments with a ligase, thereby producing a plurality of plasmids. In some embodiments, the method further includes transforming a population of microorganisms with the plasmids, wherein about 95% to 100% of the transformed microorganisms include a plasmid comprising an insert fragment and a vector fragment in a predetermined orientation.

In particular embodiments, at least 90%, at least 91%, at least 92%, at least 93%, at least 94%, at least 95%, at least 96%, at least 97%, at least 98%, at least 99%, or 100% of the transformed microorganisms include a plasmid comprising an insert fragment and a vector fragment in a predetermined orientation.

In some embodiments, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, about 99% to about 100%, or 100% of the transformed microorganisms include a plasmid comprising an insert fragment and a vector fragment in a predetermined orientation.

In certain embodiments, the method further includes purifying a plurality of the insert fragments before the ligation step.

In some embodiments, the first restriction enzyme and the second restriction enzyme are the same. In some embodiments, the first restriction enzyme and the second restriction enzyme are different.

In some embodiments, the first nucleotide sequence comprises a PCR product. In some embodiments, the first nucleotide sequence is isolated from a second plasmid.

In certain embodiments, the first nucleotide sequence comprises at least 2, at least 3, at least 4, at least 5, at least 6, at least 7, at least 8, at least 9, or more, internal sites cleaved by the first restriction enzyme. In certain embodiments, the second nucleotide sequence comprises at least 2, at least 3, at least 4, at least 5, at least 6, at least 7, at least 8, at least 9, or more, internal sites cleaved by the second restriction enzyme.

In some embodiments, the first population of nucleotide fragments comprises at least 3, at least 4, at least 5, at least 6, or more, types of insert fragments, wherein each of the 3, 4, 5, 6, or more, types of insert fragment comprises a unique non-palindromic overhang. In some embodiments, the first population of nucleotide fragments comprises at least 3, at least 4, at least 5, at least 6, or more, types of insert fragments, wherein at least 3, at least 4, at least 5, at least 6, or more, types of insert fragment comprises a unique non-palindromic overhang.

In some embodiments, the first nucleotide sequence is not methylated.

In some embodiments, the restriction enzyme is a Type IIS restriction enzyme. In some embodiments, the restriction enzyme is Bsr I, Bsm I, BsmA I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, Psr I, Ple I, Fau I, Sap I, BspQ I, BspM I, SfaN I, Hga I, Bvb I, Fok I, BceA I, BsmF I, Ksp632 I, Eco31 I, Esp3 I, Aar I, Mly I, or Btr I. In particular embodiments, the restriction enzyme is SapI.

In some embodiments, the non-palindromic overhangs of the insert fragments and the vector fragments independently include 3, 4, 5, 6, 7, 8, 9, or more nucleotides.

In another aspect, the invention features a method of producing a high efficiency cloning vector. In some embodiments, the method includes cleaving at least 2 sites of a vector sequence with at least one restriction enzyme to generate a population of vector fragments and non-vector fragments, the vector fragments comprising a non-palindromic overhang at a 5′ end, at a 3′ end, or at both. In some embodiments, the method further includes purifying the vector fragments, thereby producing a high efficiency cloning vector. In certain embodiments, at least 95% of a population of microorganisms transformed with a plasmid generated by ligating the high efficiency cloning vector and an insert nucleotide sequence comprising a corresponding non-palindromic overhang at a 5′ end, at a 3′ end, or at both, comprise a plasmid comprising the insert fragment and the high efficiency cloning vector in a predetermined orientation.

In some embodiments, at least 90%, at least 91%, at least 92%, at least 93%, at least 94%, at least 95%, at least 96%, at least 97%, at least 98%, at least 99%, or 100% of a population of microorganisms transformed with the purified plasmids include a plasmid comprising an insert fragment and a vector fragment in a predetermined orientation.

In some embodiments, about 90% to 100%, about 91% to about 100%, about 92% to about 100%, about 93% to about 100%, about 94% to about 100%, about 95% to about 100%, about 96% to about 100%, about 97% to about 100%, about 98% to about 100%, about 99% to about 100%, or 100% of a population of microorganisms transformed with the purified plasmids include a plasmid comprising an insert fragment and a vector fragment in a predetermined orientation.

In particular embodiments, one restriction enzyme cleaves at least 2 sites of the vector. In some embodiments, one restriction enzyme cleaves at least 1 site of the vector, and a second restriction enzyme cleaves at least 1 site of the vector.

In certain embodiments, the vector sequence comprises at least 2, at least 3, at least 4, at least 5, at least 6, at least 7, at least 8, at least 9, or more, internal sites cleaved by the at least one restriction enzyme.

In some embodiments, the population of vector fragments comprises at least 3, at least 4, at least 5, at least 6, or more, types of vector fragments, wherein each of the 3, 4, 5, 6, or more, types of vector fragment comprises a unique non-palindromic overhang. In some embodiments, the population of vector fragments comprises at least 3, at least 4, at least 5, at least 6, or more, types of vector fragments, wherein at least 3, at least 4, at least 5, at least 6, or more, types of vector fragment comprises a unique non-palindromic overhang.

In particular embodiments, the restriction enzyme is a Type IIS restriction enzyme. In some embodiments, the restriction enzyme is Bsr I, Bsm I, BsmA I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, Psr I, Ple I, Fau I, Sap I, BspQ I, BspM I, SfaN I, Hga I, Bvb I, Fok I, BceA I, BsmF I, Ksp632 I, Eco31 I, Esp3 I, Aar I, Mly I, or Btr I. In particular embodiments, the restriction enzyme is SapI.

In some embodiments, the non-palindromic overhangs of the vector fragments independently include 3, 4, 5, 6, 7, 8, 9, or more nucleotides.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 is a diagrammatic illustration of an exemplary cloning method.

FIG. 2 is a diagrammatic illustration of an exemplary amplification primer useful in the methods described herein.

FIG. 3 is a diagrammatic illustration of amino acid sequence analysis of Mannosidase 7.

FIG. 4 is a schematic illustration of the nucleotide sequences of a wild type vector and primers used to mutate a SapI restriction site.

FIG. 5 is a plasmid map of the pIRES cloning vector.

FIG. 6 is a plasmid map of a mutated pIRES cloning vector.

### DETAILED DESCRIPTION

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure includes novel cloning methods that are highly efficient and that enable a high percentage of the cloned sequences to be in the correct orientation. The methods utilize restriction enzymes that produce non-palindromic overhangs within double-stranded sequences of target sequences and vector sequences.

### DEFINITIONS

The articles “a” and “an” are used herein to refer to one or to more than one (i.e., to at least one) of the grammatical object of the article. By way of example, “an element” means one element or more than one element.

The term “about” is used herein to mean a value − or +20% of a given numerical value. Thus, “about 60%” means a value of between 60−(20% of 60) and 60+(20% of 60) (i.e., between 48 and 70).

As used herein, “purified” (or “isolated”) refers to a nucleic acid sequence (e.g., a polynucleotide) or an amino acid sequence (e.g., a polypeptide) that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acid sequences. An isolated nucleic acid sequence or amino acid sequence can be at least 60% free, or at least 75% free, or at least 90% free, or at least 95% free from other components present in the natural environment of the indicated nucleic acid sequence or amino acid sequence.

As used herein, “polynucleotide” (or “nucleotide sequence” or “nucleic acid molecule”) refers to an oligonucleotide, nucleotide, or polynucleotide, and fragments or portions thereof, and to DNA and RNA of genomic or synthetic origin, which may be single- or double-stranded, and represent the sense or anti-sense strand.

As used herein, “polypeptide” (or “amino acid sequence” or “protein”) refers to an oligopeptide, peptide, polypeptide, or protein sequence, and fragments or portions thereof, and to naturally occurring or synthetic molecules. “Amino acid sequence” and like terms, such as “polypeptide” or “protein”, are not meant to limit the indicated amino acid sequence to the complete, native amino acid sequence associated with the recited protein molecule.

As used herein, the terms “coupled”, “linked”, “ligated” “joined”, “fused”, and “fusion” are used interchangeably. These terms refer to the joining together of two more elements or components by whatever means, including chemical conjugation or recombinant means.

As used herein, an “amplification reaction” refers to any chemical reaction, including an enzymatic reaction, which results in increased copies of a template nucleic acid sequence.

The term “primer”, as used herein, refers to a nucleic acid sequence that primes the synthesis of a polynucleotide in an amplification reaction. A primer can comprise fewer than about 100 nucleotides and preferably comprises fewer than about 30 nucleotides. Exemplary primers range from about 5 to about 25 nucleotides.

A “target” or “target sequence”, as used herein, refers to a single or double stranded polynucleotide sequence sought to be amplified in an amplification reaction.

As used herein, “insert fragment” refers to a nucleotide sequence of interest from a target sequence. The ligation of an insert fragment in a predetermined orientation with a vector fragment yields a plasmid of interest.

As used herein, “non-insert fragment” refers to a nucleotide sequence that does not include a nucleotide of interest. A plasmid including a non-insert fragment is not a plasmid of interest.

As used herein, “vector fragment” refers to a nucleotide sequence of interest from a vector.

As used herein, “non-vector fragment” refers to a nucleotide sequence of a vector that does not include a vector sequence of interest. A plasmid including a non-vector fragment is not a plasmid of interest.

“Restriction endonuclease” and “restriction enzyme” are used interchangeably herein to refer to any of a group of enzymes, produced by bacteria, that recognize specific nucleotide sequences (“recognition sites”) and cleave molecules of DNA at the recognition site or at a distance from the recognition site. A restriction enzyme recognition site is the specific nucleotide sequence to which a restriction enzyme binds prior to cutting DNA.

As used herein, the term “overhang” refers to a portion of a strand of a double-stranded nucleic acid molecule that extends in a 5′ or a 3′ direction beyond the terminus of the complementary strand of the nucleic acid molecule.

As used herein, a “ligase” is an enzyme that ligates cleaved moieties of double-stranded DNAs by binding a 5′-phosphate end and 3′-OH end of respective adjacent DNA strands via a phosphodiester bond.

As used herein, a “predetermined orientation” or “correct orientation” refers to the ligation of one or more insert fragments with one or more vector fragments in a particular, designed manner. For example, a predetermined orientation can be one in which the insert fragment and the vector fragment are ligated in a proper translational reading frame.

In accordance with the present disclosure, there may be employed conventional molecular biology, microbiology, and recombinant DNA techniques within the skill of the art. Such techniques are described in the literature (see, e.g., Sambrook, Fritsch & Maniatis (ibid.); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

### Production of Plasmids Using High Efficiency Cloning

The methods described herein are useful for producing, with high efficiency, plasmids containing particular insert sequences in specified orientations. The high efficiency is based, in part, on the use of restriction enzymes to cleave double-stranded nucleic acids and produce non-palindromic overhangs at the 5′ and/or 3′ ends of insert sequences and also to cleave vectors to produce complementary non-palindromic overhangs of vector sequences. Ligating the digested insert and digested vector fragments to produce plasmids and transforming host cells with the plasmids results in a very high percentage (e.g., greater than about 95%) of cells containing plasmids having the desired insert and vector sequences in the correct orientation. Surprisingly, the presence of internal restriction enzyme sites within the insert sequences does not affect the high cloning efficiency.

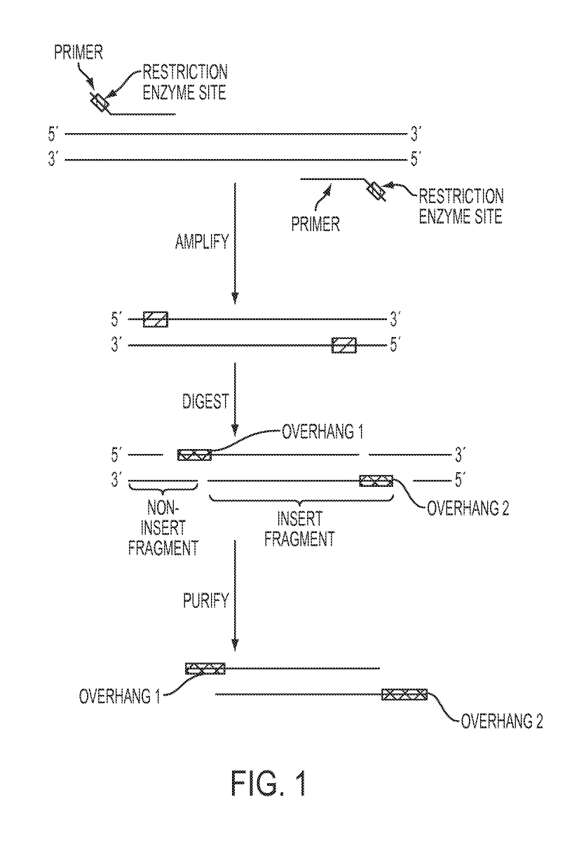

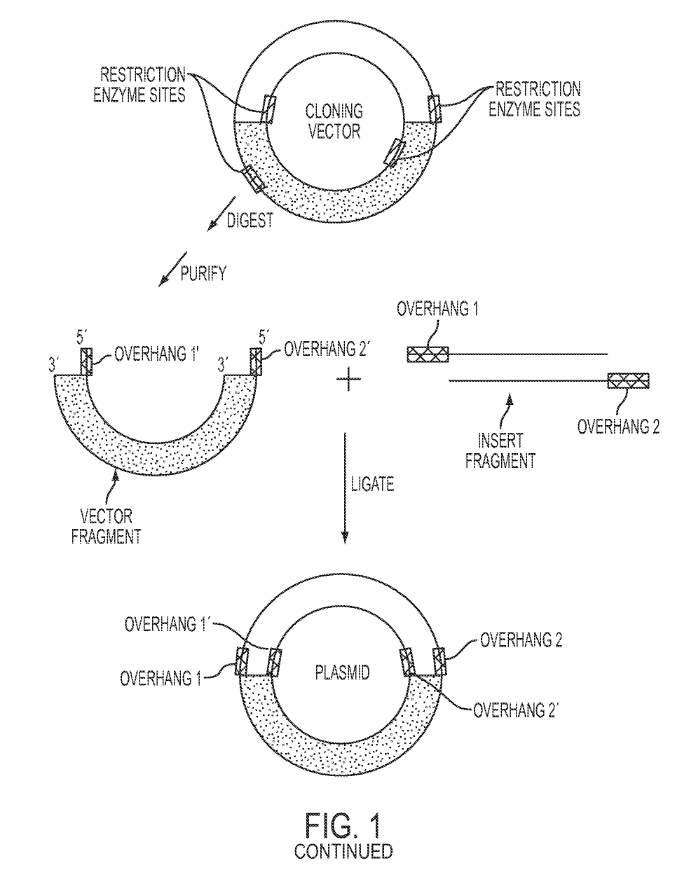

One exemplary cloning method according to the present disclosure is depicted schematically in FIG. 1. In a first step, primers are designed to hybridize to each strand of a double-stranded target sequence and are used to amplify the target sequence, e.g., by PCR. The primers include a restriction enzyme site recognized by a restriction enzyme that produces a non-palindromic overhang within each strand. Following amplification, double-stranded target sequences are produced, which include the restriction enzyme sites near the 5′ ends of each target sequence strand. Digestion with the restriction enzyme yields a double-stranded insert fragment having a non-palindromic overhang extending toward the 5′ end of each strand (“overhang 1” depicted in the top sequence and “overhang 2” depicted in the bottom sequence). Also depicted are smaller fragments (“non-insert fragments”) produced from the 5′ and 3′ ends of each amplified strand, which do not include the target sequence. Optionally, the insert fragments are purified from the non-insert fragments, resulting in purified insert fragments having overhang 1 and overhang 2, as shown in FIG. 1.

Also depicted in FIG. 1 is the digestion of a double-stranded cloning vector for insertion of an insert fragment. The particular cloning vector shown contains two restriction enzyme sites, such that digestion with the restriction enzyme results in two vector fragments, each with non-palindromic overhangs. The shaded area of the cloning vector indicates the vector fragment of interest (“vector fragment”), which can be purified from the remaining vector fragment (“non-vector fragment”). Each strand of the purified double stranded vector fragment contains a non-palindromic overhang extending toward the 5′ end of each strand (“overhang 1′” depicted in the top strand and “overhang 2”′ depicted in the bottom strand). The restriction enzyme used to cleave the cloning vector is the same one used to cleave the target sequence, such that overhang 1 of the insert fragment is complementary to overhang 1′ of the vector fragment, and overhang 2 of the insert fragment is complementary to overhang 2′ of the vector fragment.

In the final step depicted in FIG. 1, the insert fragment and the vector fragment are ligated in a directional manner by the hybridization of overhang 1 to overhang 1′ and the hybridization of overhang 2 to overhang 2′, yielding a circular plasmid that can be used for transformation or transfection of host cells.

In certain nonlimiting instances, the restriction enzyme sites included in the primers used to amplify and produce the insert fragments are different from each other. For example, each primer can include the same recognition sequence for a particular restriction enzyme and can also include unique, ambiguous nucleotides outside the recognition sequence, as described herein. In other nonlimiting instances, each primer can include a different recognition sequence, e.g., a recognition sequence for a different restriction enzyme.

In yet other nonlimiting instances, the restriction enzyme sites included in the primers used to amplify and produce the insert fragments include additional nucleotides, such as between the recognition sequence and the cleavage site for a particular restriction enzyme (e.g., for a Type-IIS restriction enzyme). In particular instances, the additional nucleotides are designed such that upon cleavage by a restriction enzyme, an overhang is produced on an insert fragment (e.g., “overhang 1” in FIG. 1) that is complementary to an overhang produced on a vector fragment (e.g., “overhang 1′” in FIG. 1). In certain instances, the sequences of the overhangs of vector fragments are predetermined, and primers used to amplify and produce the insert fragments are designed to result in complementary overhangs on insert fragments. In such instances, one vector can be used for cloning various insert fragments according to the methods described herein.

### Restriction Enzymes

Any restriction enzyme that results in non-palindromic overhangs can be used in the methods described herein. Enzymes that produce non-palindromic overhangs of, e.g., 3, 4, 5, 6, 7, or more nucleotides are useful in the methods described herein. For example, Type-IIS restriction enzymes recognize specific sequences of nucleotide base pairs within a double-stranded polynucleotide sequence. Upon recognizing that sequence, the endonuclease will cleave the polynucleotide sequence, generally leaving an overhang on one or both strands of the sequence. Type-IIS restriction enzymes generally do not recognize palindromic sequences and cleave outside of their recognition sites. For example, the Type-IIS restriction enzyme SapI recognizes and cleaves in the following manner:

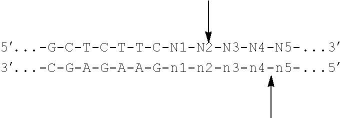

where the recognition sequence is -G-C-T-C-T-T-C; “Nx” and “nx” represent complementary, ambiguous (i.e., nonspecific) nucleotides; and the arrows indicate the cleavage sites in each strand. As this example illustrates, the recognition sequence is non-palindromic, and cleavage occurs outside of that recognition site. The cleavage occurs within an ambiguous portion of the polynucleotide sequence, and the cleavage results in a non-palindromic overhang of 3 nucleotides in each strand (i.e., “N2-N3-N4” and “n2-n3-n4”).

Because the cleavage occurs within an ambiguous portion of the polynucleotide sequence, SapI cleavage can result in different fragments having different sequences at the 3-nucleotide overhangs.

Nonlimiting examples of Type-IIs endonucleases that are useful in the present methods include, e.g., Bsr I, Bsm I, BsmA I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, Psr I, Ple I, Fau I, Sap I, BspQ I, BspM I, SfaN I, Hga I, Bvb I, Fok I, BceA I, BsmF I, Ksp632 I, Eco31 I, Esp3 I, Aar I, Mly I, and Btr I. Type-IIs endonucleases are well known in the art and are commercially available (from, e.g., New England Biolabs, Beverly, Mass.).

### Amplification of Insert Sequences

Any sequence of interest can be cloned into a vector using the methods described herein. In some embodiments, a sequence (referred to herein as an “insert fragment”) to be cloned into a vector is obtained by amplification from a target sequence. Such amplification reactions of an RNA or DNA template are known in the art. Exemplary, nonlimiting methods include polymerase chain reaction (PCR) and ligase chain reaction (LCR) (see U.S. Pat. Nos. 4,683,195 and 4,683,202; PCR Protocols. A Guide to Methods and Applications (Innis et al., eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999)) and branched DNA signal amplification (bDNA) (see, e.g., Iqbal et al., Mol. Cell Probes 13(4):315 (1999)).

In particular methods, amplification primers are used to amplify a target DNA sequence. Amplification primer pairs are designed to hybridize to a target sequence and to include a restriction enzyme recognition site near the 5′-end of each primer. The restriction endonuclease restriction site can be for the same or different endonucleases for the pair of primers. In certain instances, both primers of a primer pair include a SapI restriction site.

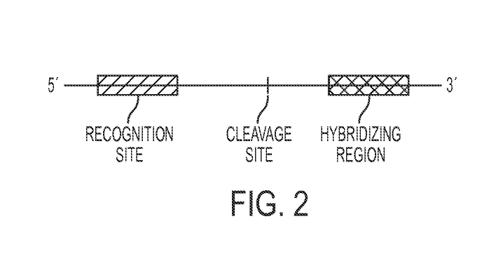

The recognition site can be placed 1, 2, 3, 4, 5, 6, 7, 8, 9, 10, 11, 12, 13, 14, 15, 16, 17, 18, 19, 20, or more, nucleotides from the 5′-end of each primer. The primers include a nucleotide sequence downstream of (i.e., 3′ to) the endonuclease cleavage site, which is capable of hybridizing with the target sequence (a “hybridizing region”). The hybridizing region of each primer can reside within a portion of the primer between a restriction endonuclease cleavage site and the 3′-end of the primer, as shown in FIG. 2. Alternatively, the hybridizing region can extend into the cleavage site and/or the 3′-end.

In some instances, one or more nucleotide mutations of a native target sequence can be included in the primer sequence between the cleavage site and the 3′-end of the primer. Such mutations can reside, for example, within the hybridizing region, as long as this region can bind to its target sequence. Sequence mutations include the addition or deletion of nucleotides, or the substitution of any given nucleotide or nucleotides. Such mutations can result in alteration of amino acid coding sequence, expression, or regulatory activity of properties of a nucleic acid segment. Those skilled in the art can easily design primers with the desired mutation(s) and without undo impairment of the priming activity of the primers used in the amplification reaction.

A suitable length of an amplification primer can be determined by one skilled in the art. For example, an amplification primer can include between about 10 and about 100 or more nucleotides, e.g., between about 15 and about 80 nucleotides, between about 15 and about 60 nucleotides, between about 15 and about 40 nucleotides, between about 15 and about 30 nucleotides, or between about 15 and about 25 nucleotides. Further, the length of the DNA containing an insert sequence is not limiting. For example, it can be about 50 to about 20000 bases, about 100 to about 10000 bases, or about 500 to about 5000 bases.

In certain embodiments, the primers are used to amplify a target sequence without including additional nucleic acids flanking the insert fragment or imposing primer sequences on the insert fragment. For example, the primers comprise a restriction enzyme recognition site for a non-palindromic restriction enzyme, allowing subsequent cleavage of the amplified product downstream of the primer sequence and proximate to the insert fragment. In such embodiments, the digested insert fragment will not contain any extraneous nucleic acids.

This amplification method is useful for any target sequence of interest, regardless of the presence of an internal site recognized by the same restriction enzyme as included in the amplification primers.

As is known to those of skill in the art, amplification reactions can be carried out in suitable reagents and, e.g., in a thermal cycler, under standard conditions to facilitate incubation times at desired temperatures. These reagents can include, but are not limited to, e.g., oligonucleotide primers described herein; borate, phosphate, carbonate, barbital, Tris, etc. based buffers (see, e.g., U.S. Pat. No. 5,508,178); salts such as potassium or sodium chloride; magnesium; deoxynucleotide triphosphates (dNTPs); a nucleic acid polymerase such as Taq DNA polymerase; DMSO; and stabilizing agents such as gelatin, bovine serum albumin, and non-ionic detergents (e.g., Tween-20).

The amplified sequence can include a target sequence of interest flanked by the restriction enzyme sites. The terminal ends of the amplified sequence can also include additional nucleotide sequence from the primers.

The amplified sequence is then digested using the restriction enzyme(s) whose recognition site was included in the amplification primers. The use of restriction enzymes to digest nucleotide sequences is known in the art and can be performed using standard conditions. The digested amplified sequence (“insert fragment”) includes a non-palindromic overhang at the 5′ end of each strand.

Amplified sequences can be purified before and/or after digestion (e.g., to purify the insert fragments from extraneous terminal nucleic acids from the primers) using conventional methods. For example, amplified sequences can be purified from agarose gels or by using a commercially available purification kit.

In other methods, insert fragments can be obtained from a vector by digesting an insert-containing vector with one or more appropriate restriction enzymes. The restriction enzymes used can produce insert fragments with suitable overhangs. Alternatively, a target sequence (i.e., a target sequence including an insert sequence of interest) can be first obtained from a vector using one or more restriction enzymes (e.g., restriction enzymes that do not result in non-palindromic overhangs), and the target sequence subsequently digested with a restriction enzyme that results in insert fragments with non-palindromic overhangs, as described herein.

### Cloning Vectors

Insert fragments described herein can be cloned into any cloning vector, including, for example, a bacteriophage such as plasmid vector DNA, a phagemid vector, a lambda phage or the like, an animal virus such as retrovirus, vaccinia virus or the like, or nucleic acids capable of self-circularization such as a cosmid. For example, a plasmid vector such as an E. coli-derived plasmid (e.g., pBR325, pUC12, pUC13), a Bacillus subtilis-derived plasmid (e.g., pUB110, pTP5, pC194), or a yeast-derived plasmid (e.g., pSH19, pSH15) can be used.

The cloning vector can be a commercially available vector, or it can readily be prepared by those skilled in the art by employing general genetic recombination techniques. Although the size of the cloning vector is not limiting, exemplary vectors are about 500 to about 20000 bases, about 1000 to about 10000 bases, or about 2000 to about 5000 bases in length.

Cloning vectors can include multiple cloning sites, which contain a variety of endonuclease restriction sites. A vector can be selected that contains one or more restriction enzyme sites that are the same as a restriction enzyme site included in the amplification primers used to generate the insert sequence. In such cases, the insert fragment and the vector fragment will have restriction cleavage sites complementary to each other. In some instances, a vector is modified using standard techniques to introduce one or more restriction enzyme sites that are compatible with a restriction enzyme used to generate the insert fragment. Specifically, the insert fragment contains an overhang that is complementary to an overhang on a vector fragment generated by digesting the vector with the restriction enzyme.

In some instances, a cloning vector has two restriction enzyme sites, and digestion with the restriction enzyme results in two fragments: a “vector fragment” into which the insert sequence will be cloned, and a “non-vector fragment”, which will not be used for cloning.

The cloning vector is digested using the restriction enzyme(s) whose recognition site was included in the amplification primers for the insert sequence. The vector fragment includes a non-palindromic overhang at the 5′ end of each strand that is complementary to the overhangs of the insert fragments. The cloning vector DNA can be purified after digestion (e.g., to purify the vector fragment from the non-vector fragment) using conventional methods, for example, using agarose gels or by using a commercially available purification kit.

### Ligation

The digested insert fragments and digested vector fragments are ligated together using methods known to those of skill in the art to produce plasmids (see, e.g., Ausubel et al. (eds.), Short Protocols in Molecular Biology, 3rd Edition (John Wiley & Sons 1995). For example, an insert fragment and a vector fragment can be ligated using a DNA ligase.

Any DNA ligase can be used in the methods described herein. Nonlimiting examples of DNA ligase useful in the methods described herein include T4 DNA ligase, T7 DNA ligase, E. coli DNA ligase, Ampligase (Epicentre Biotechnologies, Madison, Wis.), Pfu ligase (Stratagene, La Jolla, Calif.), Rma DNA ligase (Matis-Prokaria, Reykjavik, Iceland), Tsc DNA ligase (Matis-Prokaria, Reykjavik, Iceland), Tth DNA ligase (Eurogenetec, Fremont, Calif.), Taq ligase (NEB, Beverly, Mass.) and the like.

The presence of complementary non-palindromic 5′ overhangs on the insert fragments and the vector fragments results in directional cloning of the insert fragments in the correct orientation into the vector fragments.

### Transformation

After ligation of an insert fragment and a vector fragment to produce a plasmid, a suitable host cell is transformed with the plasmids and cultured, i.e., expanded, using conventional techniques. Methods for introducing the plasmids into host cells to produce host cells comprising one or more of the cloned nucleic acid molecules and/or vectors are within the skill of those in the art. Exemplary methods include infection, transduction, electroporation, transfection, and transformation. Such techniques are reviewed, for example, in Sambrook, J., et al., Molecular Cloning, a Laboratory Manual, 2nd Ed., Cold Spring Harbor, N.Y., Cold Spring Harbor Laboratory Press, pp. 16.30-16.55 (1989), and Watson, J. D., et al., Recombinant DNA, 2nd Ed., New York: W.H. Freeman and Co., pp. 213-234 (1992).

Transformed host cells can be selected, e.g., by plating and picking colonies using a standard marker, e.g., beta-galactosidase/X-gal. After selection, the vector-containing hosts are combined and expanded in cultured. The vectors are then isolated, e.g., by a conventional mini-prep, or the like, and cleaved with appropriate restriction enzymes to determine the proper insertion. The fragments are analyzed, e.g. by gel electrophoresis.

The cloning methods described herein are highly efficient cloning methods. When screening colonies, about 80% to about 100% of the colonies have the correct insert fragment(s) in the correct orientation within the vector fragment(s), e.g., about 85% to about 100%, about 90% to about 100%, about 91%, about 92%, about 93%, about 94%, about 95%, about 96%, about 97%, about 98%, about 99%, or 100% of the colonies have the correct insert fragment(s) in the correct orientation within the vector fragment(s).

In certain instances, due to the high cloning efficiency, ligation reactions are not screened for proper insertion, but the ligated plasmids are introduced directly into expression systems for polypeptide expression, such as described in Sambrook, J., et al. (ibid.). The expressed polypeptide can be validated, e.g., by transforming the initial ligation mixture into a host cell and selecting a colony. The nucleotide can by analyzed by restriction enzyme digestion as described herein, and/or the nucleotide can be sequenced using known methods.

The disclosure is further illustrated by the following example. The example is provided for illustrative purposes only. It is not to be construed as limiting the scope or content of the disclosure in any way.

### EXAMPLES

### Example 1

### High-Efficiency Plasmid Production Using Non-Palindromic Cloning

In this example, the SapI restriction enzyme was used to subclone PCR products into a cloning vector, resulting in very high cloning efficiency.

### 1. Production of Cloning Vector

pETK47b[+] (available from, e.g., EMD Biosciences, Darmstadt, Germany) was mutated to eliminate the SapI site close to the pUC-based on using the mutagenesis technique described below and then was digested with NdeI and PacI. The resulting large fragment of the vector was ligated to a double-stranded oligo that supplied dual SapI sites and other restriction enzyme sites as well as N-terminal and C-terminal his tags.

### 2. Production of Sequence Inserts

The Mannosidases listed in Table 1 below were expressed:

TABLE 1

| Polypeptide | Mol. Wt. | internal SapI sites |
|---|---|---|
| Man1 - BT2113 | 127,166 Da | 0 |
| Man2 - BT4072 | 94,501 Da | 2 |
| Man3 - BT3774 | 130,286 Da | 1 |
| Man4 - BF0339 | 130,926 Da | 0 |
| Man5 - BH0789 + | 86,533 Da | 1 |
| 2 start sites | | |
| Man7 - BH0791 | 117,652 Da | 0 |

BT = Bacteroides thetaiotaomicron
BF = Bacteroides fragilis
BH = Bacillus halodurans

Genomic DNA sequences encoding these polypeptides were amplified by PCR from genomic DNA derived from ATCC cultures of the above bacteria using standard PCR conditions. The primers used to amplify these sequences added SapI restriction enzyme sites at the 5′ and 3′ ends of the PCR products. The PCR products were then digested with SapI. To confirm the digestion of the inserts by SapI, the digests were initially analyzed by polyacrylamide gel electrophoresis (PAGE).

As shown in Table 1, Mannosidase 1, Mannosidase 4, and Mannosidase 7 do not contain internal SapI sites, Mannosidase 3 and Mannosidase 5 contain 1 internal SapI site, and Mannosidase 2 contains two internal SapI sites. The two internal SapI sites in Mannosidase 2 are shown below (recognition sites are underlined, and the resulting overhangs are in bold):

first SapI site in Mannosidase 2:
5′-AAGTTTCGCTCTTCT ACAGAAGCTCCGGCTAAGCTGA-3′
3′-TTCAAAGCGAGAAGATGT CTTCGAGGCCGATTCGACT-5′
second SapI site in Mannosidase 2:
5′-GACAAGGCTGCTATAG CCACGAAGAGCGATTGTTGG-3′
3′-CTGTTCCGACGATATCGGT GCTTCTCGCTAACAACC-5′

PAGE analysis showed that the Mannosidase 1, Mannosidase 4, and Mannosidase 7 digests resulted in a single band, the Mannosidase 3 and Mannosidase 5 digests resulted in two bands, and the Mannosidase 2 digests resulted in three bands, consistent with the predicted number of internal SapI sites for these sequences.

3. Cloning the Inserts into the Vector

PCR products were generated and digested with SapI, as described above. Following digestion, the inserts were purified using PureLink PCR Purification kit from Invitrogen (Grand Island, N.Y.) using the B3 binding buffer.

The pETKHis2 vector (described above) was digested with SapI, and was purified using the PureLink PCR Purification kit from Invitrogen using the B3 binding buffer to eliminate DNA fragments smaller than 300 nt. The DNA was eluted with water.

The purified inserts were ligated into the digested vector using ligase under standard conditions, and the ligation mixtures were transformed into NEBalpha cells (New England Biolabs, Beverly, Mass.). The transformed cells streaked onto LBKan plates and incubated at 37° C. for between 14 and 18 hours. Individual colonies were selected, and minipreps were prepared. The minipreps were digested with NdeI and HindIII restriction enzymes to remove the inserts from the vector.

PAGE analysis of the digested minipreps demonstrated high cloning efficiency. For example, of 20 colonies screened for the Mannosidase 4 insert, 19 showed the predicted band size for the insert.

Surprisingly, of 18 colonies screened for the Mannosidase 2 insert, all 18 showed the predicted band size for the insert. Thus, even when there were four DNA fragments (three Mannosidase 2 fragments and one vector fragment), this method ligated all four fragments together in the correct order at very high efficiency. This demonstrates that high cloning efficiency is achievable even when internal SapI sites are present within an insert.

### 4. Expressing Polypeptides Following High Efficiency Plasmid Production

In a separate experiment, all of the constructs described above were ligated into the pETKHis2 vector as described above. Following ligation, the plasmids were transformed without picking colonies into an expression strain. After inducing expression, they were run on a protein gel to visualize the expression and solubility. The Western blot was done using an anti-his tag antibody. The bacterial cultures were spun down and resuspended in a chemical lysis buffer, BugBuster with rLyzozyme (Novagen) and were spun again after lysis. An appropriate amount of cell lysate was run on a 4-12% SDS-Page gel for total protein staining with coomassie blue and a duplicate was blotted onto nitrocellulose and used in a Western blot using a penta-His antibody from Qiagen. The reactive proteins were visualized with a chemiluminescent substrate for the HRP enzyme attached to the antibody.

Three of the six mannosidases (Mannosidase 1, Mannosidase 3, and Mannosidase 4) were expressed reasonably well, were soluble, and were close to the predicted molecular weights. Mannosidase 5 and Mannosidase 7 were expressed well but were insoluble. Mannosidase 2 was not expressed well in this experiment.

### Example 2

### Production of Soluble Mannosidase 7

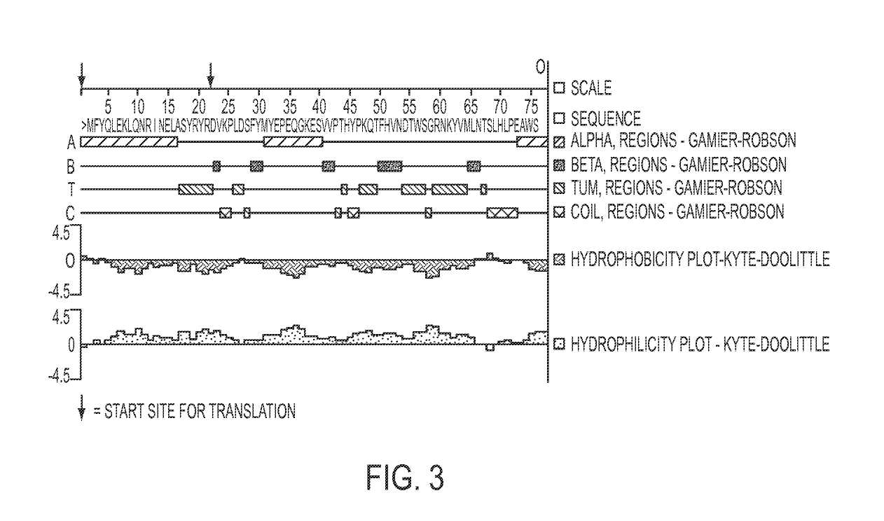

Most of the mannosidases from bacterial sources are secreted extracellularly or into the periplasmic space, but sometimes it is not clear. Originally Mannosidase 7 was thought to have a signal sequence and the PCR primers were designed to start at Asp22. However, as described in Example 1, this was not soluble. As shown in FIG. 3, new primers were made that started at Met1 (aaccgctcttccATGTTTTATCAACTGGAAAAGCTGCA).

All cells expressing different Mannosidase constructs were grown in a 24-well plate using an in-house formulation of auto-inducing media based on William Studier's, called TY media. They were lysed in Bug-Buster/rLysozyme as before. No colonies were picked prior to transformation, but the transformation mix was used directly to transform an expression strain.

12 μl of a mixture that consisted of 1 μl of SapI-digested and qualified vector plus 10 μl of Quick Ligation buffer (NEB) plus 1 μl T4 DNA ligase were added to each well used of a 96 well plate. 8 μl of a SapI-digested PCR product that contained the coding region of a protein to be expressed was added to each 12 μl mixture. The ligation was allowed to proceed for 15 to 30 minutes at approximately 23° C. 2 μl of this ligation mix was added to 20 μl of competent cells that were the expression strain to be used, such as BL21/DE3 or T7 Express (both from NEB). The cells were incubated on ice for 15 minutes, heat-shocked for 60 seconds at 42° C. and then 200 μl of pre-warmed SOC media was added and the cells were incubated with shaking at 37° C. for 30 minutes. 100 μl of these transformed cells were added to 500 μl of pre-warmed TY media with 50 μg/ml kanamycin and 0.2% lactose. After the cells were grown at either 37° C. or to an OD of about 15 AU600 the cells were spun down and resuspended completely in 80 μl resuspension buffer plus lysozyme and then 320 μl of lysis buffer plus 1 u benzonase (Novagen) was added. This mixture was incubated with shaking at RT for 20 minutes until the mixture was translucent. 15 μl of this total lysate was added to 15 μl of water plus 10 μl 4×LDS, heated at 95° C. for 5 minutes and then was spun briefly prior to running the sample on a 4-12% SDS-Page gel with or without reducing agent.

The expression levels were even better than shown in Example 1, and Mannosidase 7 was soluble, and was even more soluble using an N-his tag than a C-his tag.

### Example 3

### Mutagenesis Using Non-Palindromic Cloning Method

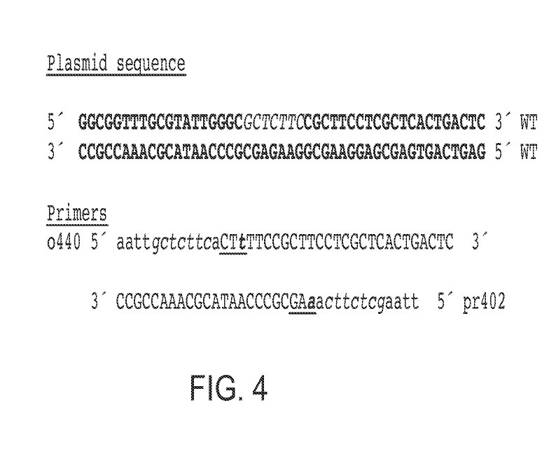

In this example, a SapI site in a plasmid was removed by mutagenesis. FIG. 4 depicts the relevant sequence of the plasmid. As shown in FIG. 4, in bold are the top and bottom strands of the sequence that were mutated, which contained an unwanted SapI recognition site (in italics). In bold and underlined are depicted the mutated bases that were used to eliminate the SapI site. Depicted by underlining are the three base overhang after cleaving the PCR product with SapI.

The two primers, o440 and pr402, were used to amplify the entire plasmid and both ends had SapI sites that were used for digestion. Once digested with SapI and ligated together, the mutants were screened by the loss of the original SapI site, and was >95% of the total colonies. The resulting plasmid contained no SapI recognition sites.

### Vector Construction

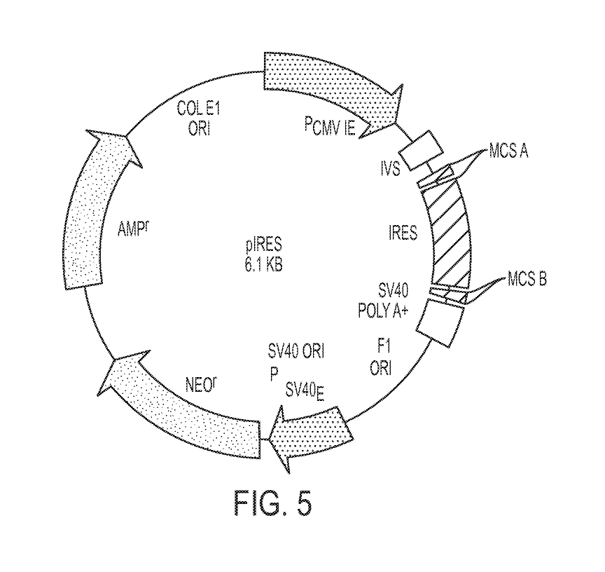

An expression vector was constructed to which a multiple cloning site that contained two SapI sites were added for use for cloning inserts. To create a mammalian expression vector, the starting vector used was pIRES, which was 6,092 bp (Clontech, see FIG. 5). To keep the plasmid as small as possible, the fl on (grey) was deleted and then DHFR (564 bp) and CTLA4-IgG (1152 bp) were added, making the final product of 7,081 bp. In addition, the second slot of the IRES element had to be altered to allow maximal expression of the DHFR gene that was placed there, and two SapI sites in the neo gene were mutated.

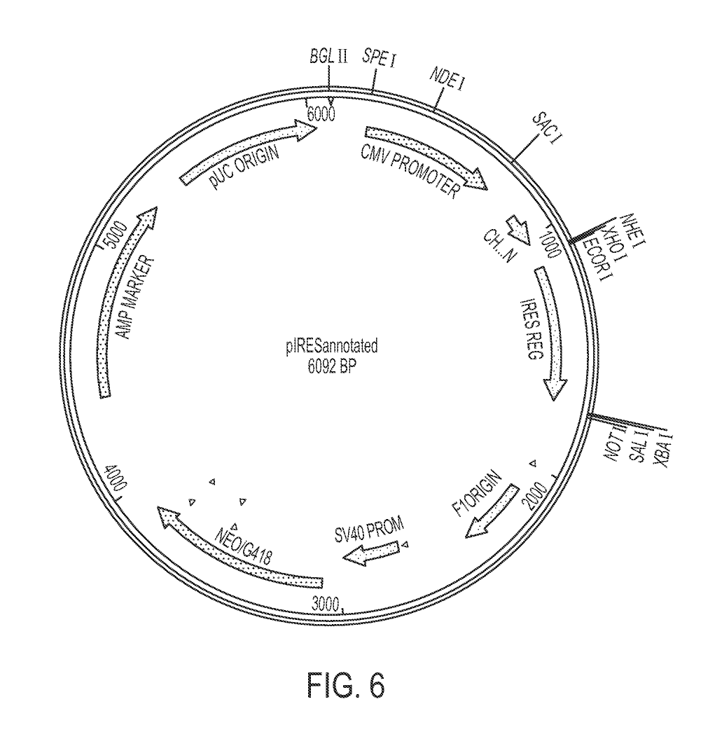

To silently mutate the two SapI sites in the neo^{r} gene of pIRES and delete the extra fl on baggage at the same time, 6 primers were used to generate 3 PCR products, which were cut with SapI and ligated back into one piece (depicted in FIG. 6). This created “pIRESm1”. Plasmids were transformed into NEBalpha or LB+Amp, colonies were isolated, and plasmid DNA was purified. The DNA was digested with NheI and SapI, and correct plasmids were screened by being linearized by NheI, but not able to be cut by SapI. Of 6 colonies screened, 5 were correct.

Using this non-palindromic cloning method, it is possible to make one or several mutations in a cloned gene or plasmid with high efficiencies, with the ending sequence being exactly what is intended. Fairly complicated vector constructs can be made very easily with this technique, where the SapI recognition sites disappear upon ligation of the various fragments. Further, this method is superior to alternative methods, which can involve using blunt end cloning, resulting in a lot of screening of clones, or using restriction sites that remain in the final construct.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing a plasmid comprising an insert fragment and a vector fragment in a predetermined orientation, the method comprising:
cleaving a first nucleotide sequence at a plurality of sites with a first restriction enzyme to generate a first population of nucleotide fragments, the first population of nucleotide fragments comprising insert fragments and non-insert fragments, the insert fragments comprising a non-palindromic overhang at a 5′ end, at a 3′ end, or at both;
cleaving a second nucleotide sequence with a second restriction enzyme to generate a second population of nucleotide fragments, the second population comprising vector fragments and non-vector fragments, the vector fragments comprising a non-palindromic overhang at a 5′ end, at a 3′ end, or at both;
purifying at least a portion of the vector fragments from the second population;
ligating a plurality of the first population of nucleotide fragments to the purified vector fragments with a ligase, thereby producing a plurality of plasmids; and
purifying the plasmids,
wherein at least 95% of a population of microorganisms transformed with the purified plasmids include a plasmid comprising an insert fragment and a vector fragment in a predetermined orientation.

2. The method of claim 1, further comprising purifying a plurality of the insert fragments before the ligation step.

3. The method of claim 1, wherein the first restriction enzyme and the second restriction enzyme are the same.

4. The method of claim 1, wherein the first restriction enzyme and the second restriction enzyme are different.

5. The method of claim 1, wherein the first nucleotide sequence comprises a PCR product.

6. The method of claim 1, wherein the first nucleotide sequence is isolated from a second plasmid.

7. The method of claim 1, wherein the first nucleotide sequence comprises at least 2 internal sites cleaved by the first restriction enzyme.

8. The method of claim 7, wherein the first population of nucleotide fragments comprises at least 3 types of insert fragments, wherein each of the 3 types of insert fragment comprises a unique non-palindromic overhang.

9. The method of claim 1, wherein the first nucleotide sequence is not methylated.

10. The method of claim 1, wherein the restriction enzyme is a Type IIS restriction enzyme.

11. The method of claim 1, wherein the restriction enzyme is SapI.

12. The method of claim 1, wherein the non-palindromic overhangs of the insert fragments and the vector fragments independently comprise 3, 4, or 5 nucleotides.

13. A method of transforming microorganisms with a plasmid comprising an insert fragment and a vector fragment in a predetermined orientation, the method comprising:
cleaving a first nucleotide sequence at a plurality of sites with a first restriction enzyme to generate a first population of nucleotide fragments, the first population of nucleotide fragments comprising insert fragments and non-insert fragments, the insert fragments comprising a non-palindromic overhang at a 5′ end, at a 3′ end, or at both;
cleaving a second nucleotide sequence with a second restriction enzyme to generate a second population of nucleotide fragments, the second population comprising vector fragments and non-vector fragments, the vector fragments comprising a non-palindromic overhang at a 5′ end, at a 3′ end, or at both;
purifying at least a portion of the vector fragments from the second population;
ligating a plurality of the first population of nucleotide fragments to the purified vector fragments with a ligase, thereby producing a plurality of plasmids; and
transforming a population of microorganisms with the plasmids,
wherein about 95% to 100% of the transformed microorganisms include a plasmid comprising an insert fragment and a vector fragment in a predetermined orientation.

14. A method of producing a high efficiency cloning vector, the method comprising:
cleaving a vector sequence with a restriction enzyme at 2 sites to generate a population of vector fragments and non-vector fragments, the vector fragments comprising a non-palindromic overhang at a 5′ end, at a 3′ end, or at both; and
purifying the vector fragments, thereby producing a high efficiency cloning vector,
wherein at least 95% of a population of microorganisms transformed with a plasmid generated by ligating the high efficiency cloning vector and an insert nucleotide sequence comprising a corresponding non-palindromic overhang at a 5′ end, at a 3′ end, or at both, comprise a plasmid comprising the insert fragment and the high efficiency cloning vector in a predetermined orientation.


SEQUENCE LISTING (11 sequences)

SEQ ID NO: 1 - DNA, 12 bp, Artificial Sequence
  Type-IIS restriction enzyme Sap1 5′-3′ sense strand recogniton site
gctcttcnnnnn

SEQ ID NO: 2 - DNA, 12 bp, Artificial Sequence
  Type-IIS restriction enzyme Sap1 5′-3′ anti- sense strand recogniton site
nnnnngaagagc

SEQ ID NO: 3 - DNA, 37 bp, Artificial Sequence
  first SapI site on sense strand in Mannosidase 2
aagtttcgctcttctacagaagctccggctaagctga

SEQ ID NO: 4 - DNA, 37 bp, Artificial Sequence
  first SapI site on anti-sense strand in Mannosidase 2
tcagcttagccggagcttctgtagaagagcgaaactt

SEQ ID NO: 5 - DNA, 36 bp, Artificial Sequence
  second SapI site on sense strand in Mannosidase 2
gacaaggctgctatagccacgaagagcgattgttgg

SEQ ID NO: 6 - DNA, 36 bp, Artificial Sequence
  second SapI site on anti-sense strand in Mannosidase 2
ccaacaatcgctcttcgtggctatagcagccttgtc

SEQ ID NO: 7 - DNA, 38 bp, Artificial Sequence
  Chemically synthesized oligonucleotide primer
aaccgctcttccatgttttatcaactggaaaagctgca

SEQ ID NO: 8 - DNA, 47 bp, Artificial Sequence
  Chemically synthesized oligonucleotide
ggcggtttgcgtattgggcgctcttccgcttcctcgctcactgactc

SEQ ID NO: 9 - DNA, 47 bp, Artificial Sequence
  Chemically synthesized oligonucleotide
gagtcagtgagcgaggaagcggaagagcgcccaatacgcaaaccgcc

SEQ ID NO: 10 - DNA, 39 bp, Artificial Sequence
  Chemically synthesized oligonucleotide
aattgctcttcacttttccgcttcctcgctcactgactc

SEQ ID NO: 11 - DNA, 35 bp, Artificial Sequence
  Chemically synthesized oligonucleotide
ttaagctcttcaaagcgcccaatacgcaaaccgcc